(19) United States
(12) Patent Application Publication
(10) Pub. No.: US 2021/0378937 A1
(43) Pub. Date: Dec. 9, 2021

(54) Methods and Compositions for Improving the Appearance of Hair Following Topical Minoxidil Application

(71) Applicant: Applied Biology, Inc., Irvine, CA (US)

(72) Inventors: Ofer A. Goren, Irvine, CA (US); John A. McCoy, Irvine, CA (US)

(21) Appl. No.: 16/946,158

(22) Filed: Jun. 8, 2020

(51) Int. Cl. A61K 8/73; A61Q 5/02; A61K 8/49; A61K 8/31; A61Q 7/00; A61K 8/04

(52) U.S. Cl. CPC A61K 8/732; A61Q 5/02; A61K 8/046; A61K 8/31; A61Q 7/00; A61K 8/494

(57) ABSTRACT

Compositions and methods are disclosed herein for treating female pattern hair loss with a topical composition used to remove minoxidil from the hairs. A method for diagnosing subjects in need of a topical composition used to remove minoxidil from the hair treatment and guiding the application of a topical composition used to remove minoxidil from the hair treatment are also described. Additionally, a method for diagnosing a subject at high risk for female pattern hair loss who will likely respond to topical composition used to remove minoxidil from the hair prophylactic treatment is described. A method for combining the composition described herein with topical minoxidil for androgenetic alopecia is described. In addition, cosmetic compositions are described as well as methods of improving the appearance of hair following application of minoxidil.

### FIELD

The present invention relates to methods and compositions for the treatment of female and male pattern hair loss. Topical compositions are described as well as a method of treatment for improving the clinical efficacy of minoxidil. In addition, cosmetic compositions are described as well as methods of improving the appearance of hair after application of topical drugs.

### BACKGROUND

In 1988 the US FDA approved 2% topical minoxidil solution as an OTC drug for the treatment of androgenetic alopecia (AGA). Since the FDA approval, minoxidil has become the mainstay therapy for AGA. However, the effectiveness of minoxidil in the general population is low, only 39% of patients respond to the drug (See Olsen E A, Whiting D, Bergfeld W, Miller J, Hordinsky M, Wanser R, et al. A multicenter, randomized, placebo-controlled, double-blind clinical trial of a novel formulation of 5% minoxidil topical foam versus placebo in the treatment of androgenetic alopecia in men. J Am Acad Dermatol. 2007 57(5): 767-74). In the pivotal study submitted to the US FDA in support of the efficacy of the 5% topical minoxidil foam, no subjects had great improvement, 8% of the subjects had a moderate improvement, and 31% of the subjects had a slight improvement (See US FDA Application 21-812 Medical Review).

2% topical minoxidil is the only US FDA approved drug for the treatment of female pattern hair loss (FPHL) also termed female androgenetic alopecia. It is estimated that 13-20% of FPHL patients experience a moderate increase in hair regrowth when using a 2% minoxidil mono-therapy. 5% minoxidil solutions are also used as a treatment for FPHL. Clinical trials suggest that 5% minoxidil has better efficacy than 2% minoxidil based on the average change in nonvellus hair count. The risk for adverse events with both 2% and 5% minoxidil is low; however, irritant dermatitis, allergic contact dermatitis, and hypertrichosis have been reported. Due to the prolonged treatment time required to elicit a therapeutic response (approximately 6 months) combined with the variable efficacy of minoxidil in the general population, an adjuvant to increase the clinical efficacy of topical minoxidil would have significant clinical utility.

For most topical drugs, compliance with the daily treatment regimen is a major barrier that limits clinical efficacy. In clinical trial, it was reported that 40% of patients prescribed topical minoxidil stopped using the drug after one month of use. Because it takes at least 3 month to see appreciable results with topical minoxidil, researchers concluded that efficacy was not the main determining factor limiting patient compliance. A survey of 300 patients who stopped using minoxidil after 1 month described the residue left on the hair after use of the product as their main objection. Subjects comments collected during the trial included descriptions of the product as greasy (62%) and sticky (46%). 58% of respondents reported that topical minoxidil “made it hard to style [their] hair normally”. As such, an adjuvant product that would improve the cosmetic aesthetics of topical minoxidil could greatly improve patient compliance with the treatment regimen and subsequently improve its clinical efficacy.

### SUMMARY

Compositions and methods are disclosed herein for treating male and female pattern hair loss with a minoxidil adjuvant therapy. Additionally, cosmetic compositions are disclosed herein with accompanying methods of improving the appearance of hair after application of topical drugs. The main function of the adjuvant therapy is to remove residue of the drug (or its vehicle) from the hair (where treatment is not needed) while leaving intact the drug on the scalp (where treatment is required). The methods and composition described can be applied as adjuvant therapy to minoxidil and would lead to increased patient compliance with the treatment regimen and hence improve its clinical efficacy.

In an exemplary embodiment, a dry shampoo, comprises: aluminum starch octenylsuccinate; wherein the dry shampoo is applied to the hair of the scalp of a person after an application of minoxidil to remove minoxidil from the hair but not the scalp.

In some embodiment, the dry shampoo comprises an exfoliating agent.

In an exemplary embodiment, a composition for the removal of minoxidil from hair while leaving minoxidil on the scalp, wherein the composition comprises a propellant and a dry shampoo, wherein the dry shampoo comprises: a modified starch; a carrier material; and a solvent; wherein the modified starch contributes 2% to 20% towards the total weight of the dry shampoo; wherein the carrier material contributes between 0.1% and about 50% towards the total weight of the dry shampoo and the propellant, and wherein the propellant contributes 25% to 90% towards the total weight of the dry shampoo and the propellant.

In some embodiments, the modified starch is aluminum starch octenylsuccinate.

In some embodiments, the composition further comprises an exfoliating agent.

In an exemplary embodiment, a method for removing an ingredient from hair while leaving the ingredient on the scalp involves: applying the ingredient to the scalp and hair growing from the scalp; applying a dry shampoo containing aluminum starch octenylsuccinate to the hair and the scalp; wherein the ingredient includes any one or combination of minoxidil, propylene glycol, ethanol, butylhydroxytoluene, stearyl alcohol, cetyl alcohol, alcohol SD, citric acid, polysorbate 60, Aeropin 70, propane, butane, and isobutene.

In some embodiments, the dry shampoo includes an exfoliating agent.

In an exemplary embodiment, a method for reducing hair greasiness after the application of minoxidil involves applying an embodiment of the dry shampoo discloses herein to the hair after treatment with minoxidil, wherein the dry shampoo does not remove minoxidil from the skin of the scalp.

In some embodiments, the method involves applying the dry shampoo 5 minutes, 10 minutes, 30 minutes, 1 hr, 2, hrs, 4 hrs, or anytime thereafter minoxidil is applied to the scalp.

In an exemplary embodiment a method to increase hair manageability after the application of minoxidil involves applying an embodiment of the dry shampoo discloses herein to the hair after treatment with minoxidil, wherein the dry shampoo does not remove minoxidil from the skin of the scalp.

In some embodiments, the method involves applying the dry shampoo 5 minutes, 10 minutes, 30 minutes, 1 hr, 2, hrs, 4 hrs, or anytime thereafter minoxidil is applied to the scalp.

In an exemplary embodiment, a method to improve the appearance of hair after the application of minoxidil involves applying an embodiment of the dry shampoo discloses herein to the hair after treatment with minoxidil, wherein the dry shampoo does not remove minoxidil from the skin of the scalp.

In some embodiments the method involves applying the dry shampoo 5 minutes, 10 minutes, 30 minutes, 1 hr, 2, hrs, 4 hrs, or anytime thereafter minoxidil is applied to the scalp.

In an exemplary embodiment, a method to reduce the stickiness of hair after the application of minoxidil involves applying an embodiment of the dry shampoo discloses herein to the hair after treatment with minoxidil, wherein the dry shampoo does not remove minoxidil from the skin of the scalp.

In some embodiments, the method involves applying the dry shampoo 5 minutes, 10 minutes, 30 minutes, 1 hr, 2, hrs, 4 hrs, or anytime thereafter minoxidil is applied to the scalp.

In an exemplary embodiment, a kit for treating or preventing alopecia in a subject in need thereof includes: a topical composition including minoxidil; and the dry shampoo of claim 1.

The kit of claim 16, wherein the dry shampoo is applied 5 minutes, 10 minutes, 30 minutes, 1 hr, 2, hrs, 4 hrs, or anytime thereafter minoxidil is applied to the scalp.

In some embodiments, the dry shampoo is applied routinely thereafter minoxidil is applied.

### DETAILED DESCRIPTION

Androgenetic alopecia (AGA) is a common dermatological condition affecting approximately 50% of the population by the age of 50. Currently, the only drug approved by the US Food and Drug Administration (FDA) for the treatment of AGA in both men and women is topical minoxidil. Clinical trials have demonstrated that following 16 weeks of 5% minoxidil therapy approximately 30-40% of patients regrow hair. Many patients discontinue using minoxidil after only a few weeks because the drug product leaves residue in their hair.

As used herein, the terms “prevent” or “prevention” and other derivatives of the words, when used in reference to alopecia, e.g., androgenetic alopecia, refer to a reduced likelihood of alopecia in an individual receiving a given treatment relative to that of a similar individual at risk for alopecia but not receiving that treatment. As such, the terms “prevent” and “prevention” encompass a treatment that results in a lesser degree of alopecia, e.g., androgenetic alopecia, than would be otherwise expected for a given individual. Efficacy for prevention of alopecia, e.g., androgenetic alopecia, can be established through controlled studies, e.g., in which a subject is administered a treatment (e.g., a topical treatment) and another subject is administered a placebo. Under these circumstances, if the subject treated with the topical treatment undergoes less hair loss over time relative to the subject receiving the placebo, e.g., at least 5% less, at least 10% less, at least 15% less, at least 20% less, at least 25% less, at least 30% less, at least 35% less, at least 40% less, at least 45% less, at least 50% less or beyond, the treatment is effective for the prevention of alopecia, e.g., androgenetic alopecia.

As used herein, the terms “treat,” “treatment,” or “treating” refer to therapeutic treatments, wherein the object is to reverse, alleviate, ameliorate, inhibit, slow down or stop the progression or severity of a disease or condition, e.g., androgenetic alopecia or other form of alopecia. The term “treating” includes reducing or alleviating at least one adverse effect or symptom of a disease or condition, e.g., androgenetic alopecia or other form of alopecia. Treatment is generally “effective” if one or more symptoms are reduced. Alternatively, treatment is “effective” if the progression of a disease is reduced or halted. That is, “treatment” includes not just the improvement of symptoms, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality. For example, treatment is considered effective if the extent or amount of hair loss is reduced, or the progression of hair loss is slowed or halted. The term “treatment” of a disease also includes providing relief from the symptoms or side-effects of the disease (including palliative treatment).

As used herein the term “comprising” or “comprises” is used in reference to compositions, methods, etc. refers to component(s) or method steps that are present in the method or composition, yet allows for the composition, method, etc. to also include unspecified elements.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

As used herein the term “alopecia” refers to all forms of hair loss in men and women including but not limited to traction alopecia, androgenetic alopecia, male pattern baldness (MPB), female pattern hair loss (FPHL), alopecia areata, alopecia universalis, telogen effluvium, chemotherapy induced alopecia, hair shedding, eyebrow hair loss, beard hair loss and hair thinning. The term permits the presence of elements that do not materially affect the basic and novel or functional characteristic(s) of that embodiment.

As used herein the term “minoxidil” refers to the minoxidil drug product solution or foam containing but not limited to 2% (w/w) minoxidil, 5% (w/w) minoxidil, or 10% (w/w) minoxidil and any one or a combination of the following used to compose the drug product: propylene glycol, ethanol, water, butylhydroxytoluene (BHT), stearyl alcohol, cetyl alcohol, alcohol SD 40B, lactic acid, citric acid anhydrous, glycerol anhydrous, polysorbate 60, propellant Aeropin 70 (propane, butane, isobutane). The term “minoxidil” can also refer to the vehicle of the drug or any ingredient in the minoxidil drug product

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

As used herein, the term “cosmetic composition” refers to any composition that can be applied to improve the appearance, texture, sculpting ability, styling, density or feel of hair. Cosmetic compositions are not limited to, but include the topical compositions disclosed herein.

Disclosed herein are methods to treat or prevent various forms of alopecia, e.g. female pattern hair loss or androgenetic alopecia. These methods include the use of a topical composition applied to hair after the application of minoxidil to remove the minoxidil from the hair while leaving minoxidil on the scalp.

Disclosed herein are methods to clean propylene glycol from hair. These methods include the use of a topical composition applied to hair after the application of minoxidil to remove propylene glycol from the hair while leaving propylene glycol on the scalp.

Disclosed herein are methods to clean ethanol from hair. These methods include the use of a topical composition applied to hair after the application of minoxidil to remove ethanol from the hair while leaving ethanol on the scalp.

Disclosed herein are methods to clean butylhydroxytoluene (BHT) from hair. These methods include the use of a topical composition applied to hair after the application of minoxidil to remove butylhydroxytoluene (BHT) from the hair while leaving butylhydroxytoluene (BHT) on the scalp.

Disclosed herein are methods to clean stearyl alcohol from hair. These methods include the use of a topical composition applied to hair after the application of minoxidil to remove stearyl alcohol from the hair while leaving stearyl alcohol on the scalp.

Disclosed herein are methods to clean cetyl alcohol from hair. These methods include the use of a topical composition applied to hair after the application of minoxidil to remove cetyl alcohol from the hair while leaving cetyl alcohol on the scalp.

Disclosed herein are methods to clean alcohol SD 40B from hair. These methods include the use of a topical composition applied to hair after the application of minoxidil to remove alcohol SD 40B from the hair while leaving alcohol SD 40B on the scalp.

Disclosed herein are methods to clean lactic acid from hair. These methods include the use of a topical composition applied to hair after the application of minoxidil to remove lactic acid from the hair while leaving lactic acid on the scalp.

Disclosed herein are methods to clean citric acid from hair. These methods include the use of a topical composition applied to hair after the application of minoxidil to remove citric acid from the hair while leaving citric acid on the scalp.

Disclosed herein are methods to clean glycerol from hair. These methods include the use of a topical composition applied to hair after the application of minoxidil to remove glycerol from the hair while leaving glycerol on the scalp.

Disclosed herein are methods to clean polysorbate 60 from hair. These methods include the use of a topical composition applied to hair after the application of minoxidil to remove polysorbate 60 from the hair while leaving polysorbate 60 on the scalp.

Disclosed herein are methods to clean Aeropin 70 from hair. These methods include the use of a topical composition applied to hair after the application of minoxidil to remove Aeropin 70 from the hair while leaving Aeropin 70 on the scalp.

Disclosed herein are methods to clean propane from hair. These methods include the use of a topical composition applied to hair after the application of minoxidil to remove propane from the hair while leaving propane on the scalp.

Disclosed herein are methods to clean butane from hair. These methods includes the use of a topical composition applied to hair after the application of minoxidil to remove butane from the hair while leaving butane on the scalp.

Disclosed herein are methods to clean isobutane from hair. These methods include the use of a topical composition applied to hair after the application of minoxidil to remove isobutane from the hair while leaving isobutane on the scalp.

Disclosed herein are cosmetic and topical compositions for the removal of minoxidil from the hair. In preferred embodiments, the compositions do not remove minoxidil from the scalp or skin of the scalp.

In one embodiment, a topical composition used to remove minoxidil from the hair is applied to a skin section, such as a section of the scalp, which contains at least one hair follicle. In another embodiment, a topical composition used to remove minoxidil from the hair is applied to the scalp, which contains at least one hair follicle to treat androgenetic alopecia or FPHL.

The therapeutic agent, particularly the topical composition used to remove minoxidil from the hair, described herein and used in the present methods may be formulated into compositions according to the knowledge of one of skill in the art. In one embodiment, the topical composition used to remove minoxidil from the hairs is formulated as a topical spray. In another embodiment the topical composition used to remove minoxidil from the hair is formulated as a dry shampoo.

The therapeutic agents, particularly the topical composition used to remove minoxidil from the hairs, described herein and used in the present methods may be formulated into compositions according to the knowledge of one of skill in the art. In one embodiment, the topical composition used to remove minoxidil from the hair is encapsulated in order to increase the water solubility of the therapeutic agent. In another embodiment, the topical composition used to remove minoxidil from the hair is encapsulated in order to reduce the loss through degradation of therapeutic agent, for example, to reduce oxidation of the therapeutic agent.

In one embodiment aluminum starch octenylsuccinate is encapsulated to overcome its poor water solubility and facile oxidative degradation.

In another embodiment, the topical composition used to remove minoxidil from the hair is formulated in a shampoo, a foam, ointment, spray, solution, gel, slow release capsule, oral tablet, dry shampoo, aerosol or any similar compound or delivery vehicle or methodology. Topical application is preferred. In one embodiment, the composition is formulated in a topical cream. In another embodiment, the composition is formulated in a hair styling product selected from the group consisting of a styling gel, a styling foam, and a hair conditioner. In one embodiment the topical composition is formulated as a dry shampoo.

In one embodiment, the formulation disclosed herein is an aerosol composition comprising a dry shampoo and a propellant. In another embodiment the composition is formulated for application as a foam or mousse. By “dry shampoo” is meant a formulation comprising a carrier material that is a volatile liquid and therefore evaporates and a powder that remains, wherein the powder contains a starch or modified starch, such as aluminum starch octenyl succinate. In other embodiments, the dry shampoo comprises a carrier material and a modified starch. Examples of suitable carrier materials that are volatile liquids are lower alcohols including without limitation ethanol or isopropanol, a volatile silicone compound such as polydimethylsiloxanes (e.g., having a viscosity less than about 5 cSt at 25° C.), cyclomethicone, cyclohexane siloxane, decamethyltetrasiloxane, octamethyltrisiloxane, decamethylpentasiloxane, decamethylcyclopentasiloxane, octamethylcyclotetrasiloxane, trimethyl silylamodimethicone, phenyl trimethicone, hexamethyidisiloxane, and dimethylsiloxane/methylalkylsiloxane, and combinations thereof. Other carrier materials known to those skilled in the art may also be used.

The total percentage weight of the carrier material in the aerosol composition may be between about 0.1% and about 50%, between about 0.1% and about 40%, between about 1% and about 35%, between about 5% and about 50%, between about 10% and about 40%, between about 15% and about 40%, of the total weight of the aerosol dry shampoo composition (a combination of the dry shampoo composition and the propellant). In certain embodiments, the composition is substantially free of water.

In one embodiment, the dry shampoo further comprises a solvent, which may or may not be volatile. The solvent can be an alcohol, such as a polyhydric alcohol. Non-limiting examples of polyhydric alcohols include 1,3-butylene glycol, propylene glycol, glycol 200 (PEG 200), polyethylene glycol 400 (PEG 400), hexylene glycol and dipropylene glycol, and glycerol. The polyhydric alcohol can have a concentration of 10% or less by weight, or 5% or less by weight, or between 10% to 1% by weight. In one embodiment, the solvent can comprise benzyl alcohol. In another embodiment, the solvent comprises propylene glycol and/or water. Low levels of water (such as 1-5% or less than 10%) are preferred in the non-foaming aerosol compositions of the invention. If the solvent is non-volatile, the solvent contributes less than 20%, less than 10%, less than 5% or less than 1% of the total weight of the aerosol dry shampoo composition (a combination of the dry shampoo composition and the propellant).

In another embodiment, the dry shampoo further comprises an acid selected from the group consisting of hydrochloric acid, sulphuric acid, nitric acid, phosphoric acid, citric acid, acetic acid, lactic acid, succinic acid, and maleic acid, and mixtures thereof.

The dry shampoo of the present embodiments may further comprise a starch or modified starch. The starch or modified starch preferably acts as a sebum absorber. Non-limiting examples of suitable starch materials include cornstarch, potato starch, tapioca starch, rice starch, wheat starch, and cassaya starch. A starch material may be modified or unmodified. A modified starch material is a starch which has been derivatized or altered by processes known to those of ordinary skill in the art, such as esterification, etherification, oxidation, acid hydrolysis, crosslinking, or enzyme conversion. Non-limiting examples of suitable modified starch materials include aluminum starch octenylsuccinate, sodium starch octenylsuccinate, calcium starch octenylsuccinate, distarch phosphate, hydroxyethyl starch phosphate, hydroxypropyl starch phosphate, sodium carboxymethyl starch, and sodium starch glycolate. In an embodiment, the dry shampoo comprises aluminum starch octenyl succinate and tapioca starch.

In other embodiments, the dry shampoo further comprises another oil-absorbing powder, for example, cellulose, chalk, talc, fuller's earth, etc. In some embodiments, a further sebum absorber is added, including a clay material, which may be stearalkonium hectorite. In some embodiments, the further sebum absorber is at least one modified clay material selected from the group consisting of stearalkonium hectorite, stearalkonium bentonite, quaternium-18 bentonite, and quaternium-18 hectorite. In some embodiments, silica is added and may function as an oil-absorbing compound and/or a suspending agent. In another embodiment, the composition is substantially free of silica and silica-containing components.

The starch material may be present in the dry shampoo at a concentration of 1% to 70% by weight, 1% to 60% by weight, 1% to 50% by weight, 1% to 40% by weight, 1% to 30% by weight, 1% to 20% by weight, 1% to 15% by weight, 1% to 10% by weight, 5% to 50% by weight, 5% to 40% by weight, 5% to 30% by weight, 5% to 20% by weight, 5% to 10% by weight, 5% to 15% by weight, 10% to 60% by weight, 10% to 50% by weight, 10% to 40% by weight, 10% to 30% by weight, 10% to 20% by weight, 10% to 15% by weight, or 20% to 60% by weight as measured based on the total weight of the dry shampoo.

In certain embodiments, the aerosol composition comprises a dry shampoo having as a starch or modified starch aluminum starch octenylsuccinate at a concentration in the dry shampoo of 2% to 20% by weight, 2% to 10% by weight, 10% to 20% by weight, 11% to 20% by weight, 10% to 15% by weight, 14 to 16% by weight, 2% to 10% by weight, 2 to 3% by weight, 3% to 4% by weight, 5% to 6% by weight, 7% to 8% by weight, 9% to 10% by weight, 11% to 12% by weight, 13% to 14% by weight, 13% to 15% by weight, 15% to 16% by weight, 17% to 18% by weight, 13% to 16% by weight, 14% to 16% by weight, 19% to 20% by weight, 2 to 8% by weight, 2 to 5% by weight, 4 to 8% by weight, or 5 to 10% by weight, or at a concentration of about 5%, 6%, 7%, 8%, 9%, 10%, 11%, 12%, 13%, 14%, 15%, 16%, 17%, 18%, 19%, or 20% by weight.

The aerosol compositions described herein may also include a propellant. Non-limiting examples of suitable propellants include butane, isobutane, propane, A-46 (isobutane and propane), liquefied petroleum gas (e.g., propane), dimethyl ether, methyl ethyl ether, trichlorofluoromethane, dichlorodifluoromethane, dichlorotetrafluorothane, monochlorodifluoromethane, trichlorotrifluoroethane propane, carbon dioxide, nitrous oxide, 1,1,1,2,-tetrafluoroethane, 1,1,2,3,3,3-heptafluoropropane, or combinations thereof. In a particular embodiment, the propellant is isobutane. A propellant may condense to a liquid state in an aerosol container at ambient temperatures. In some embodiments, the propellant may have a lower specific gravity as compared to the rest of the composition, thus facilitating propelling the composition from a container (e.g., through a dip tube) as compared to expelling the propellant.

The propellant may be present at a concentration of 25% to 90% by weight, 25% to 80% by weight, 25% to 70% by weight, 25% to 60% by weight, 25% to 50% by weight, 25% to 40% by weight, 25% to 30% by weight, 30% to 90% by weight, 30% to 80% by weight, 30% to 70% by weight, 30% to 60% by weight, 30% to 50% by weight, 30% to 40% by weight, 40% to 90% by weight, 40% to 80% by weight, 40% to 70% by weight, 40% to 60% by weight, 40% to 50% by weight, 50% to 90% by weight, 50% to 80% by weight, 50% to 70% by weight, or 50% to 60% by weight of the total composition (dry shampoo and propellant).

In one embodiment, the aerosol composition comprises about 2% to about 20%, or about 2% to about 10% by weight of aluminum starch octenylsuccinate. This composition may further comprise alcohol, propylene glycol, and water. In another embodiment, the aerosol composition comprises a propellant and a dry shampoo, wherein the dry shampoo comprises aluminum starch octenyl succinate at a concentration of 2%-10% by weight, SD alcohol 40-B, and polysorbate 60. In another embodiment, the aerosol composition comprises a propellant and a dry shampoo, wherein the dry shampoo comprises 2%-20% by weight of aluminum starch octenyl succinate, ethanol, and an acid, wherein the composition has an apparent pH between 5.0 and 7.0. The dry shampoo may further comprise about 1%-5% by weight of magnesium stearate. In one embodiment, the aerosol composition comprises a propellant and a dry shampoo, wherein the dry shampoo comprises 2%-20% by weight of aluminum starch octenyl succinate, ethanol, an acid, acrylates copolymer and magnesium carbonate, wherein the combined concentration of acrylates copolymer and magnesium carbonate is 5%-15% by weight. This embodiment may further comprise a propellant containing dimethyl ether. In another embodiment, the dry shampoo comprises 10%-17% by weight of aluminum starch octenyl succinate, ethanol, an acid at an apparent pH between 6.0 and 6.5, and 5%-15% by weight of a combination of acrylates copolymer and magnesium carbonate. This embodiment may further comprise about 1%-5% by weight of magnesium stearate. In a further preferred embodiment, the dry shampoo comprises about 14% by weight of aluminum starch octenyl succinate, ethanol, lactic acid at an apparent pH between 6.0 and 6.5, and 5-15% by weight of a combination of acrylates copolymer and magnesium carbonate.

In another embodiment, the aerosol composition comprises a propellant and a dry shampoo, wherein the dry shampoo comprises aluminum starch octenyl succinate at a concentration of 2%-20% by weight, ethanol, and propylene glycol. In this embodiment, the composition may be substantially free of water. It may further comprise in the dry shampoo acrylates copolymer and magnesium carbonate. A suitable propellant for this embodiment is dimethyl ether.

In one embodiment, the aerosol composition comprises a propellant and a dry shampoo, wherein the dry shampoo comprises about 13%-17% by weight of aluminum starch octenyl succinate, ethanol, about 3%-6% by weight of magnesium stearate, and an acid at an apparent pH between 5.0 and 7.0.

In another embodiment, the dry shampoo comprises 13-15% by weight of aluminum starch octenyl succinate, ethanol, an acid at an apparent pH between 6.0 and 6.5, and 5-15% by weight of a combination of acrylates copolymer and magnesium carbonate. In one embodiment, the dry shampoo may further comprise about 1%-5% by weight of magnesium stearate. In yet another embodiment, the dry shampoo comprises about 16% by weight of aluminum starch octenyl succinate, ethanol, lactic acid at an apparent pH between 6.0 and 6.5, and 5-15% by weight of a combination of acrylates copolymer and magnesium carbonate. In one embodiment, the dry shampoo further comprises about 5% by weight of magnesium stearate.

In another embodiment, the aerosol composition comprises a propellant and a dry shampoo, wherein the dry shampoo comprises aluminum starch octenyl succinate at a concentration of 2%-20% by weight, tapioca starch, SDA-40-2, and an acid. In this embodiment, the propellant may comprise A-46 (15.2% propane, 84.8% isobutene), propane and/or isobutene.

In one embodiment, the aerosol composition comprising a propellant and a dry shampoo, wherein the dry shampoo comprises about 2%-20% or about 6% to about 20% by weight of aluminum starch octenyl succinate and a solvent. Alternatively, in this embodiment, the dry shampoo comprises about 2%-10% by weight of aluminum starch octenyl succinate. In another alternative, the dry shampoo further comprises about 2%-5% by weight of magnesium stearate, and/or is substantially free of water. In another variation of this embodiment, the dry shampoo comprises about 13%-17% by weight of aluminum starch octenyl succinate.

In another embodiment, the aerosol composition comprises a propellant and a dry shampoo, wherein the dry shampoo comprises about 13%-17% by weight of aluminum starch octenyl succinate, about 2%-7% by weight tapioca starch, SDA-40-2, and an acid at an apparent pH between 5.0 and 7.0. In one embodiment, the dry shampoo further comprises about 2%-5% by weight of magnesium stearate. In one embodiment, the composition furthermore is substantially free of water. In another embodiment, the dry shampoo comprises about 14-16% by weight of aluminum starch octenyl succinate, about 4%-6% by weight tapioca starch, SDA-40-2, and an acid at an apparent pH between 6.0 and 6.5. In one embodiment, the dry shampoo further comprises about 2%-4% by weight of magnesium stearate. In a further preferred embodiment, the dry shampoo comprises about 15% by weight of aluminum starch octenyl succinate, about 6% by weight of tapioca starch, SDA-40-2, and lactic acid at an apparent pH between 6.0 and 6.5.

In another embodiment, the aerosol composition comprising a propellant and a dry shampoo, wherein the dry shampoo comprises 2%-20% by weight of aluminum starch octenyl succinate, about 4%-6% by weight tapioca starch, SDA-40-2, and an acid at an apparent pH between 5.0 and 7.0.

In another embodiment, the aerosol composition comprises a solvent of a lower alcohol, wherein the composition is not formulated for application as a foam or mousse. This composition may further include a modified starch, and further comprises a propellant that is present at a concentration of 25-90% by weight of the total weight of the aerosol composition. It may further comprise at least one additive component, such as polysorbate 60. Preferably, the foregoing composition is stored in a non-combustible propellant delivery system, which may be a bag-on-valve system. In another embodiment, the aerosol composition comprises a propellant, a modified starch such as aluminum starch octenylsuccinate, SD alcohol 40-B and optionally polysorbate 60 and/or lactic acid, wherein the composition is substantially free of water.

In one embodiment of the present invention a composition is formulated to remove minoxidil from the hair while leaving minoxidil on the scalp. In one embodiment the composition may contain isobutane, propane, denatured alcohol, aluminum starch octenylsuccinate, isopropyl myristate, silica, fragrance, or Argania spinosa Kernel Oil.

In one embodiment of the present invention a composition is formulated to remove minoxidil from the hair while leaving minoxidil on the scalp. In one embodiment the composition may contain corn starch, rice starch, Oryza sativa starch, cetrimonium chloride, aluminum starch, silica, activated carbon, alumina, anionic surfactants, clay, or Rhassoul clay.

In another embodiment, the compositions may comprise an exfoliating agent to promote absorption of the topical composition used to remove minoxidil from the hair into scalp. It is contemplated for the exfoliating agent to be salicylic acid; however, other exfoliating agents can be used. Other examples of the exfoliating agents include (1) inorganic and/or metallic particles such as: boron nitride, in body-centered cubic form (Borazon®); aluminosilicate (e.g. nepheline); zircon; mixed oxides of aluminum such as emery; zinc oxide; aluminum oxides such as aluminas or corundum; titanium oxide; titanium oxide coated mica; carbides, in particular silicon carbide (carborundum); or other metal oxides; metals, and metal alloys such as iron shot, steel shot, and in particular perlite; silicates such as glass, quartz, sand, or vermiculite; calcium carbonate (e.g. Bora-Bora sand or Rose de Brignoles sand) or magnesium carbonate; sodium chloride; pumice stone; amorphous silica; diamond; ceramics, and (2) organic particles such as: fruit stones, in particular apricot stones, e.g. Scrubami® apricot; wood cellulose, e.g. ground bamboo stem; coconut shell, e.g. coconut exfoliator; polyamides, in particular Nylon-6; sugars; plastic microbeads, e.g. polyethylenes or polypropylenes; ground walnut; ground apricot seed; ground shells, and (3) mixed particles associating organic and inorganic compounds, and particles coated in the above compounds. The exfoliating agents may be in the form of microbeads of less than five millimeters in its largest dimension that have an exfoliating effect.

The compositions disclosed herein may be formulated into a drug. In preferred embodiments, the compositions used to remove minoxidil from the hair can be formulated as a cosmetic product.

In another embodiment, a cosmetic composition is used to remove from hair the vehicle used in a topical minoxidil drug composition.

In one embodiment, a cosmetic composition disclosed herein is applied to the hair after minoxidil is applied and has dried on the scalp.

In another embodiment, a cosmetic composition disclosed herein is applied to the hair 5 minutes, 10 minutes, 30 minutes, 1 hr, 2, hrs, 4 hrs or anytime thereafter minoxidil is applied to the scalp.

Disclosed herein is also a method for reducing hair greasiness after the application of minoxidil, wherein a cosmetic composition as disclosed herein is applied to the hair after treatment with minoxidil. The application of the cosmetic composition may occur either 5 minutes, 10 minutes, 30 minutes, 1 hr, 2, hrs, 4 hrs or anytime thereafter minoxidil is applied or when minoxidil has dried on the scalp.

Disclosed herein is also a method to increase hair manageability (or ease of styling) after application of minoxidil, wherein a cosmetic composition as disclosed herein is applied to the hair after treatment with minoxidil. The application of the cosmetic composition may occur either 5 minutes, 10 minutes, 30 minutes, 1 hr, 2, hrs, 4 hrs or anytime thereafter minoxidil is applied or when minoxidil has dried on the scalp.

Disclosed herein is also a method to improve the appearance of hair after application of minoxidil, wherein a cosmetic composition as disclosed herein is applied to the hair after treatment with minoxidil. The application of the cosmetic composition may occur either 5 minutes, 10 minutes, 30 minutes, 1 hr, 2, hrs, 4 hrs or anytime thereafter minoxidil is applied or when minoxidil has dried on the scalp.

Disclosed herein is also a method to reduce the stickiness of hair after application of minoxidil, wherein a cosmetic composition as disclosed herein is applied to the hair after treatment with minoxidil. The application of the cosmetic composition may occur either 5 minutes, 10 minutes, 30 minutes, 1 hr, 2, hrs, 4 hrs or anytime thereafter minoxidil is applied or when minoxidil has dried on the scalp

The compositions used in the present disclosure, particularly compositions used to remove minoxidil from the hair, may be formulated with a preservative such as EDTA (0.1-0.5% by weight of the formulation) and/or sodium metabisulfite (0.1-0.5% by weight of the formulation). In some embodiments, a penetration enhancer is selected from one or more of the group consisting of alcohols, glycols, fatty acids, fatty esters, fatty ethers, occlusive agents, surface active agents, dimethylaminopropionic acid derivatives, terpenes, sulfoxides, cyclic ethers, amides, and amines. Other components of the formulations used herein may be chosen from cosmetically approved excipients known in the art, including water, thickeners, etc.

The composition may be packaged in a kit with an applicator for application to the hair. The invention is also directed to a kit comprising a composition of the therapeutic agent, such as a topical composition used to remove minoxidil from the hair, and an applicator, and to a kit comprising a composition of the therapeutic agent, such as a topical composition used to remove minoxidil from the hair, and a hair brush or comb, particularly a brush or comb that provides exfoliating effect on the scalp such that there is light abrasion after its use that enhances penetration of the therapeutic in the scalp. In one embodiment, the therapeutic agent is provided in a metered dose applicator that provides for a fixed volume of the composition to be administered with each administration, such as 1 ml of the topical composition per administration. It is specifically envisioned that the composition used to remove minoxidil from the hair may be packaged as a kit with minoxidil.

The composition may be packaged in a kit including a topical minoxidil formulation. For example, a 2% minoxidil topical solution, a 3% topical minoxidil solution, a 5% topical minoxidil solution, a 5% topical minoxidil foam, a 10% topical minoxidil solution.

It will be understood that the ranges described above, and throughout this document, are also intended to encompass single values contained within these ranges. For example, for a formulation comprising a particular ingredient in a range between 1-50%, a percentage of 5% or 49% is also intended to be disclosed.

Any of the aforementioned topical composition used to remove minoxidil from the hair can be used routinely, e.g., once daily, twice daily, every other day, once a week. Routine use of the topical composition used to remove minoxidil from the hair would be indicated as an adjuvant therapy for minoxidil in androgenetic alopecia patients. In is specifically envisioned that a composition (e.g., a dry shampoo) of any of the aforementioned topical composition used to remove minoxidil from the hair can be used daily by a person using minoxidil to increase the effectiveness of minoxidil.

Efficacy of treatment to treat or prevent androgenetic alopecia can be determined by monitoring the density of hairs on a given area of the subject's body, e.g., a given area of the scalp. If the rate of hair loss is reduced, e.g., by 10% or more following treatment, the treatment is effective for the prevention of androgenetic alopecia. Similarly, if hair density remains the same, the treatment is effective for the prevention of androgenetic alopecia. If the density of hair increases, e.g., by 5% or more, e.g., by 10% or more following treatment, the treatment is also considered effective for the treatment and/or prevention of androgenetic alopecia.

Efficacy of treatment to treat or prevent androgenetic alopecia can be determined by monitoring global photography. For example, the patient or an expert can assess the treatment response utilizing before and after global photographs.

As noted above, it is contemplated that all forms of alopecia can benefit from the technology described herein. For example, the technology described herein can be applicable to prevent or treat androgenic alopecia.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that can have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

### EXAMPLES

### Example 1: Study to Evaluate Patient Satisfaction with Minoxidil Kit

Overview: 42 female subjects were recruited to a placebo controlled study to assess the likelihood that each subject would continue to use a kit composed of a minoxidil and a dry shampoo formulated to remove minoxidil residue from the hair.

Method: 42 FPHL patients were recruited from the PIs patient pool. Subjects were brought to the PIs office and instructed to apply a 5% minoxidil solution according to the instructions on the product label. After application of the minoxidil product each patient was instructed to use an after minoxidil product which was either the formula being tested or a placebo. After the application of the second product patients were asked to complete a survey to assess their experience and whether or not they would likely continue to use the kit on a regular basis.

Efficacy Endpoints:

1. Before minoxidil application ratings:
Hair manageability easiness 1..10 (1 easy to manage)
Hair greasiness 1..10 (1 not greasy)
Hair styling easiness 1..10 (1 easy)
2. After minoxidil application ratings:
Hair manageability easiness 1..10 (1 easy to manage)
Hair greasiness 1..10 (1 not greasy)
Hair styling easiness 1..10 (1 easy)
3. After Formula or Placebo application Rating:
Hair manageability easiness 1..10 (1 easy to manage)
Hair greasiness 1..10 (1 not greasy)
Hair styling easiness 1..10 (1 easy)

Formulas

Inventive Example: Dry shampoo containing isobutane, propane, denatured alcohol, aluminum starch octenylsuccinate, isopropyl myristate, silica, fragrance, Argania spinosa kernel oil SDS-IL-2096.

Comparative Example (Placebo): Dry shampoo containing isobutane, propane, denatured alcohol.

Results:

| | Formula | Placebo |
|---|---|---|
| 1. Before Minoxidil | | |
| Manageability | 5.25 | 5.64 |
| Greasiness | 6.25 | 6.10 |
| Ease of Styling | 4.13 | 4.70 |
| 2. After Minoxidil | | |
| Manageability | 7.25 | 8.20 |
| Greasiness | 8.50 | 7.63 |
| Ease of Styling | 6.38 | 6.58 |
| 3. After Formula or | | |
| Placebo | | |
| Manageability | 4.63 | 7.93 |
| Greasiness | 4.50 | 7.00 |
| Ease of Styling | 3.50 | 7.23 |

Additionally, subjects in both arms were asked the likelihood that they would continue to used the minoxidil kit on a daily basis. 18 of 22 subjects in the active arm responded that they would likely continue to used the minoxidil kit versus 6 of 20 subjects in the placebo arm.

### Example 2

A study was conducted to assess the effect of a dry shampoo containing aluminum starch octenylsuccinate on minoxidil efficacy. 60 female pattern hair loss subjects were recruited to the study. In one arm, 30 subjects applied daily for 24 weeks a 2% minoxidil solution followed by application of the dry shampoo (5 minutes after applying minoxidil). In the other arm, 30 subjects applied daily for 24 weeks a 2% minoxidil solution alone. At the end of the study both groups exhibited similar rates of hair growth i.e., 14.1%+/−4.7 and 13.9%+/−4.8; therefore, it can be concluded that the dry shampoo does not interfere with the efficacy of minoxidil.

1. A dry shampoo, comprising:
aluminum starch octenylsuccinate;
wherein the dry shampoo is applied to the hair of the scalp of a person after an application of minoxidil to remove minoxidil from the hair but not the scalp.

2. The dry shampoo of claim 1, further comprising an exfoliating agent.

3. A composition for the removal of minoxidil from hair while leaving minoxidil on the scalp, wherein the composition comprises a propellant and a dry shampoo, wherein the dry shampoo comprises:
a modified starch;
a carrier material;
and a solvent;
wherein the modified starch contributes 2% to 20% towards the total weight of the dry shampoo;
wherein the carrier material contributes between 0.1% and about 50% towards the total weight of the dry shampoo and the propellant, and
wherein the propellant contributes 25% to 90% towards the total weight of the dry shampoo and the propellant.

4. The composition of claim 3, wherein the modified starch is aluminum starch octenylsuccinate.

5. The composition of claim 3, further comprising an exfoliating agent.

6. A method for removing an ingredient from hair while leaving the ingredient on the scalp, the method comprising:
applying the ingredient to the scalp and hair growing from the scalp;
applying a dry shampoo containing aluminum starch octenylsuccinate to the hair and the scalp;
wherein the ingredient includes any one or combination of minoxidil, propylene glycol, ethanol, butylhydroxytoluene, stearyl alcohol, cetyl alcohol, alcohol SD, citric acid, polysorbate 60, Aeropin 70, propane, butane, and isobutene.

7. The method of claim 6, wherein the dry shampoo includes an exfoliating agent.

8. A method for reducing hair greasiness after the application of minoxidil, the method comprising applying a dry shampoo comprising aluminum starch octenylsuccinate to the hair after treatment with minoxidil, wherein the dry shampoo does not remove minoxidil from the skin of the scalp.

9. The method of claim 8, further comprising applying the dry shampoo 5 minutes, 10 minutes, 30 minutes, 1 hr, 2, hrs, 4 hrs, or anytime thereafter minoxidil is applied to the scalp.

10. A method for increase hair manageability after the application of minoxidil, the method comprising applying the dry shampoo of claim 8 to the hair after treatment with minoxidil, wherein the dry shampoo does not remove minoxidil from the skin of the scalp.

11. The method of claim 10, further comprising applying the dry shampoo 5 minutes, 10 minutes, 30 minutes, 1 hr, 2, hrs, 4 hrs, or anytime thereafter minoxidil is applied to the scalp.

12. A method to improve the appearance of hair after the application of minoxidil, the method comprising applying the dry shampoo of claim 8 to the hair after treatment with minoxidil, wherein the dry shampoo does not remove minoxidil from the skin of the scalp.

13. The method of claim 12, further comprising applying the dry shampoo 5 minutes, 10 minutes, 30 minutes, 1 hr, 2, hrs, 4 hrs, or anytime thereafter minoxidil is applied to the scalp.

14. A method to reduce the stickiness of hair after the application of minoxidil, the method comprising applying the shampoo of claim 8 to the hair after treatment with minoxidil, wherein the dry shampoo does not remove minoxidil from the skin of the scalp.

15. The method of claim 14, further comprising applying the dry shampoo 5 minutes, 10 minutes, 30 minutes, 1 hr, 2, hrs, 4 hrs, or anytime thereafter minoxidil is applied to the scalp.

16. A kit for treating or preventing alopecia in a subject in need thereof, comprising:
a topical composition including minoxidil; and
the dry shampoo of claim 1.

17. The kit of claim 16, wherein the dry shampoo is applied 5 minutes, 10 minutes, 30 minutes, 1 hr, 2, hrs, 4 hrs, or anytime thereafter minoxidil is applied to the scalp.

18. The kit of claim 16, wherein the dry shampoo is applied routinely thereafter minoxidil is applied.
